(19) United States
(12) Patent Application Publication
(10) Pub. No.: US 2026/0326992 A1
(43) Pub. Date: Oct. 22, 2026

(54) Puncture-Resistant Protected Inner Container Device

(71) Applicant: Brigitte Paola Cardona, Deerfield, FL (US)

(72) Inventor: Brigitte Paola Cardona, Deerfield, FL (US)

(21) Appl. No.: 19/553,270

(22) Filed: Feb. 28, 2026

Related U.S. Application Data
(60) Provisional application No. 63/765,413, filed on Feb. 28, 2025.

(51) Int. Cl. B65D 77/04; A61B 5/15; B65D 81/02

(52) U.S. Cl. CPC B65D 77/0493; A61B 5/150343; A61B 5/150351; B65D 81/022

(57) ABSTRACT

A puncture-resistant protected inner container comprising an outer protective body, an inner glass container, a rubber stopper having an upper portion and a lower portion, the lower portion being frictionally introduced into the top opening of the inner glass container to form a fluid-tight seal, introduced into the outer protective body and secured therein by an outer closure cap. The stopper comprises a centrally disposed puncture site. In one embodiment, the outer closure cap is introduced over the upper portion with the puncture site being accessible. In one embodiment, the outer closure cap further comprises an outer top rim and a puncture site, such that when the outer closure cap is frictionally introduced over the upper portion, the outer top rim rest on the top surface of the upper portion, such that the puncture sites are in alignment.

### CROSS-REFERENCE TO RELATED APPLICATIONS

This application claims priority from U.S. Provisional Application 63/765,413 filed Feb. 28, 2025, the disclosure of which is incorporated herein by reference.

### FIELD OF INVENTION

The present invention relates to a healthcare equipment and puncture-resistant protected inner container devices, specifically a glass inner container suitable for use for blood collection, blood separation, and the processing of blood components. More particularly, the present invention relates to a device designed for protecting breakable glass containers, such as blood collection glass tubes, which are capable of holding fluid and clotted specimens for storage, analysis, and/or processing in pharmaceutical, clinical, forensic, dental, industrial, agricultural, environmental, medical, surgical, and veterinary applications.

### BACKGROUND OF THE INVENTION

For many years, in the biomedical science and healthcare industries, blood has been drawn to perform various tests to assess a human's health, as well as an animal's health. In certain instances, test tubes for storing, transporting, and processing blood may contain an additive or a surface coat for promoting or inhibiting clotting, depending upon the intended application. From the collected blood sample, the intended application may require the testing or processing of serum, plasma, or plasma in a fibrin clot. To accomplish this application, the blood is collected and stored in a test tube composed of glass, particularly a “plain glass blood collection tube.” In other instances, the blood is collected and stored in a plastic test tube, such as polyethylene terephthalate (PET), with the addition of a chemical additive or coating.

Traditionally, blood collection tubes were made from glass, particularly until the mid-1990s. Specifically, these blood collection tubes were composed of non-reactive glass. An advantage of these glass tubes in blood collection is that glass tubes do not require a clotting additive or silica coat because the natural surface of the glass tube inherently promoted clotting. Thus, blood contained in a glass tube would coagulate, and during centrifugation, blood separation happens and the red components of the blood fall to the bottom of the tube, leaving a plasma clot and a supernatant of serum at the upper position of the tube.

Glass test tubes and glass blood collection tubes are ideal for biomedical and health care applications requiring the retrieval of plasma clots or serum. For example, newer regenerative treatments with platelet-rich plasma (PRP) and platelet-rich fibrin (PRF), require drawing a person's blood, spinning the sample down in a centrifuge for plasma separation, retrieving plasma in PRP or clotted plasma in PRF and supernatant, and treating the person with the plasma clot and supernatant that may or may not be further processed to mix with bone graft materials and improve bone graft handling characteristics. These applications are distinguished from merely drawing blood for in vitro health screenings because part of the collected sample that is platelet-rich is returned to a person's body.

More recently, modern blood collection tubes and test tubes have been made from PET-medical grade plastic which requires treatment with additives and/or coatings, especially for applications requiring centrifugation, to obtain the separation of serum, or a plasma clot and plasma supernatant, from whole blood. Indeed, plastic is almost exclusively used due to the risks posed with glass test tubes and glass collection tubes, including breakage, contamination of the sample, contamination of the laboratory, contamination and injury of the personnel handling the sample in the glass tube, and the spread of blood-borne pathogens and diseases. As a result, test and blood collection tube materials, additives, coatings, and specifications now are regulated by governing agencies such as the U.S. Food and Drug Administration (USFDA), NIOSH, CDC, and OSHA, which produced a joint advisory notice requiring the use of test and blood collections tubes that are not manufactured from glass unless such glass tubes are puncture resistant protected.

A significant advantage of using a plain glass test tube is that it does not require the use of an additive or silica coat in applications requiring the retrieval of plasma clots or serum. Conversely, plastic (PET) test tubes generally are treated with silicone, additives, and/or a silica coat. One serious disadvantage of such PET tubes is that inhalation and internal exposure to silica may pose potential occupational-exposure risks. Further, exposure to the additives likely poses an industrial occupational hazard for those in the manufacturing sector. Individuals receiving treatments with their own serum or plasma are exposed to the additive and/or silica coat residue. In some instances, this exposure is directly into the bloodstream if the serum or plasma is injected or topically applied to an open wound or suture. In other instances, FDA regulations require that PRP, PRF and other autologous blood concentrates must be free to additives and coats that could affect blood purity.

For example, the use of platelet-rich fibrin (PRF) applications in dental procedures is becoming increasing popular, mixing with bone graft materials to improve bone grafting handling characteristics that can lead to expedited healing. The PRF procedure activates a clotting cascade to obtain a the PRF clot. This activation occurs naturally in glass tubes. When using a PET tube, a silica coat is required to activate the clotting cascade and obtain the PRF clot.

Other autologous blood plasma separation is PRP that uses PET tubes to which have been added anticoagulants to obtain plasma separation and used to mix with bone graft handling characteristics. Additional applications for PRP include hair loss, wrinkle and antiaging, and orthopedic treatments for expedited healing. These treatments typically require multiple sessions or multiple blood collection tubes. Consequently, these multiple sessions and/or multiple tubes result in greater exposure from the additives used in plastic (PET) test tubes.

Accordingly, sample exposure to additives inherently occurring with plastic (PET) tubes treated with additives or silica coats should be avoided while still conforming to government regulations for blood components'purity and safety.

### SUMMARY OF THE INVENTION

The invention of the present subject matter relates to a puncture-resistant protected inner container device comprising an outer protective body, a breakable blood collection glass tube, hereinafter sometimes referred to as an inner glass container or a glass tube, a rubber stopper having an upper portion and a lower portion, the upper portion and lower portion being separated by a stopper groove, the lower portion of the rubber stopper being introduced into and frictionally secured within the upper opening of the inner glass container to create a vacuum seal, and an outer closure cap which is configured to be introduced over and frictionally engage the upper portion of the rubber stopper and the upper portion of the inner glass container to create a second seal, and removably coupled within the top sleeve of the outer protective body, such as by frictional engagement to create a third seal. This frictional securing arrangement provides enhanced stability and contamination prevention while protecting the contents of the inner glass container. It also creates a second seal between the outer closure cap and the upper portion of the inner glass container. The outer protective body is composed of a, puncture-resistant material which is adapted to retain the inner glass container and configured to provide a third seal as well as positional stabilization of the inner glass container within the outer protective body. The bottom end of the outer protective body further may comprise a stabilizing base in the form of a surface-engaging stabilization structure, suitable examples of which include, a ring-shaped support base, a suction cup stabilization element configured to releasably engage a support surface, and an elastomeric support base, such as a plurality of silicone feet.

Accordingly, it is an object of the present subject matter to provide a cost-effective, durable, and puncture-resistant protected inner container device for a glass blood collection tube at least partially filled with a blood sample that activates clotting without the use of a silica coat.

It is another object of the present subject matter to provide a puncture-resistant protected inner container device for a blood collection glass tube which is adapted to contain a sample including, for example, a bodily fluid, such as blood or serum, a soil or water sample, a pharmaceutical preparation, or a nutritional product.

It is still another object of the present subject matter to provide a puncture-resistant protected inner container device, comprising an outer protective body and an inner glass container in the form of a blood collection glass tube, the inner glass container being configured to be disposed within the internal cavity of the outer protective body.

It is a yet another object of the present subject matter to provide a puncture-resistant protected inner container device for an inner glass container having rubber stopper which prevents contamination or expulsion of a sample disposed therein, the rubber stopper having a lower portion being frictionally secured within the upper opening of the inner glass container.

It is a further object of the present subject matter to provide a puncture-resistant protected inner container device comprising an inner glass container in the form of a blood collection glass tube having a rubber stopper with an upper portion and a lower portion, the upper portion and lower portion separated by a stopper groove, the lower portion being introduced into and frictionally secured within the upper opening of inner glass container, and an outer protective body having an outer closure cap which is introduced over and frictionally engages the upper portion of the rubber stopper and the upper portion of the inner glass container to form a second seal, and removably coupled within the top sleeve of the outer protective body by frictional engagement.

It is yet a further object of the present subject matter to provide a puncture-resistant protected inner container device comprising an outer protective body, an inner glass container, a rubber stopper in the form of a rubber cap seal having a top surface with a centrally disposed puncture site, a lower portion and an upper portion separated by a stopper groove, the lower portion of rubber stopper being introduced into and frictionally secured within the upper opening of the inner glass container, and an outer closure cap configured with an opened outer cap top end having an outer cap top end rim with, and an opened outer cap bottom end, the opened bottom end being introduced over the upper portion of the rubber stopper and the upper portion of the inner glass container to form a second seal, and removably coupled within the top sleeve of the outer protective body by frictional engagement in such a manner that the top end of the outer closure cap extends downwardly over the upper portion of the rubber stopper and outer cap top end rim is flush with the top surface of the of the rubber, thus making the puncture site being fully accessible.

It is still a further object of the present subject matter to provide a puncture-resistant protected inner container device comprising an outer protective body, an inner blood collection glass tube, a rubber stopper in the form of a rubber cap seal having a top surface with a centrally disposed puncture site, the lower portion of rubber stopper being frictionally secured within the upper end opening of the glass tube, and an outer closure cap, the outer closure cap configured with an outer cap top end having an outer cap top end rim and a puncture site opening centrally disposed within the outer cap top end, and an open outer cap bottom end, the open bottom end being introduced over the upper portion of the rubber stopper and removably coupled within the top sleeve of said outer protective body by frictional engagement in such a manner that the outer closure cap extends downwardly over the upper portion of the rubber stopper and over the upper portion of the inner glass container in frictional engagement to form a second seal, the outer cap top end rim contacting the top surface of the rubber stopper with the centrally disposed puncture sites of the rubber stopper and the outer closure cap in alignment and accessible.

It is another object of the present subject matter to provide a puncture-resistant protected inner container device which can be disposed in a single sterile pack and be configured for an intended application, such as platelet-rich fibrin applications in dental and oral surgery procedures, as well as any other medical and surgical procedures where platelet-rich fibrin applications can be beneficial.

It is also an object of the present subject matter to provide a puncture-resistant protected inner container device having an outer protective body with a stabilizing base to add stability to the bottom end of the outer protective body, examples of which include a ring-shaped support base, a suction cup stabilization element configured to releasably engage a support surface, and an elastomeric support base, such as a plurality of silicone feet disposed at the bottom end of the outer protective body.

It is a further object of the present subject matter to provide a puncture-resistant protected inner container device which provides multiple safety and quality-control advantages over the conventional breakable container systems including (1) mitigation of breakage risk during handling, transport, and centrifugation, (2) reduction of puncture or laceration injury associated with glass fracture, (3) containment of glass fragments upon accidental breakage, (4) protection of sample integrity, (5) non-destructive inspection capability, and (6) validation of glass rim integrity prior to use.

It is also an object of the present subject matter to provide a puncture-resistant protected inner container device having selective exposure of the upper rim of the inner glass container which enables non-destructive integrity verification, thereby mitigating the risks associated with comprised vacuum seals or rim fractures as the upper rim of the inner glass container represents a critical structural zone susceptible to chips, cracks, or fracture initiation.

These and other objects of the present subject matter are accomplished by providing a puncture-resistant protected inner container device comprising an inner glass container disposed within an internal cavity of an outer protective body composed from a polymeric or elastomeric material, including for example, silicone, silicone-containing materials, polypropylene, polyethylene terephthalate (PET), and the like. This outer protective body is removably coupled by frictional contact to the inner glass container, without any communication that allows fluid or air passage between the inner container and outer protective body.

### BRIEF DESCRIPTION OF DRAWINGS

FIG. 1 is an exploded view of the puncture-resistant protected inner container device of the present subject matter having an outer closure cap which frictionally slides over the rubber stopper such that the top surface of the rubber stopper extends upwardly from the outer closure cap.

FIG. 2 is a side perspective view of the assembled puncture-resistant protected inner container device of FIG. 1, depicting the top surface of the rubber stopper flush with the top end outer closure cap, the puncture site of the rubber stopper being fully accessible.

FIG. 3 is an exploded view of the puncture-resistant protected inner container device of the present subject matter having an outer closure cap which frictionally slides over the rubber stopper, the outer closure cap having an upper outer rim which contacts the top surface of the rubber stopper.

FIG. 4 is a top perspective view of the assembled puncture-resistant protected inner container device of FIG. 3 illustrating the alignment of the puncture sites of the rubber and the outer closure cap.

FIG. 5 is a side view of the outer protective body of the puncture-resistant protected inner container device of the present subject matter depicting a plurality of elastomeric silicone feet as the stabilizing base.

FIG. 6A is a cross-sectional exploded view of the puncture-resistant protected inner container device of the present subject matter

FIG. 6B is a cross-sectional view of the assembled puncture-resistant protected inner container device of the present subject matter.

FIG. 7 is a perspective view of the protected blood collection glass tube of the present subject matter packaged in a single-use, sterile pack.

It is to be understood that the drawings are not necessarily to scale. Rather, the drawings are merely representations, not intended to portray specific parameters of the present subject matter unless specified otherwise. The drawings are intended to depict exemplary embodiments of the present subject matter, and therefor should not be considered limiting in scope. In the drawings, like numbering represents like elements. Further, certain elements in some figures may be omitted, or not illustrated to scale, for illustrative clarity. Any cross-sectional views may be in the form of “slices”, or “near-sighted” cross-sectional views, omitting certain background lines that may otherwise be visible in a “true” cross-sectional view, for illustrative clarity. In some instances, where multiple elements are present, only a single element may be labeled or multiple elements may have the same label to indicate that the label applies to all elements of the same type.

### DETAILED DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

The terms “inner container,” “inner tube,” “inner glass tube,” “blood collection glass tube,” derivatives thereof and similar terms may be used throughout the specification and it is to be understood that such terms are interchangeable and all refer to an inner glass container. Similarly, the terms “elastomeric container,” “silicone protected body,” “outer body,” “outer protective body,” “protective device,” derivatives thereof and similar terms may be used throughout the specification and it is to be understood that such terms are interchangeable and all refer to the protective outer body of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

Reference throughout this specification to “one embodiment” or “an embodiment” means that a particular feature, structure, or characteristic described in connection with the embodiment is included in at least one embodiment. Thus, the use of the phrases “in one embodiment” or “in an embodiment” throughout the specification are not necessarily referring to the same embodiment. Further, particular features, structures, or characteristics may be combined in any suitable manner in one or more embodiments. Headings provided herein are for convenience only and do not interpret the scope or meaning of the claimed invention. The present disclosure is directed to overcoming or at least reducing the effects of one or more of the problems presented above.

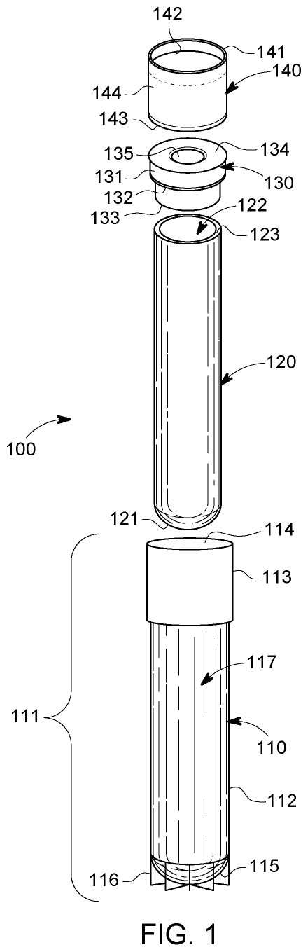

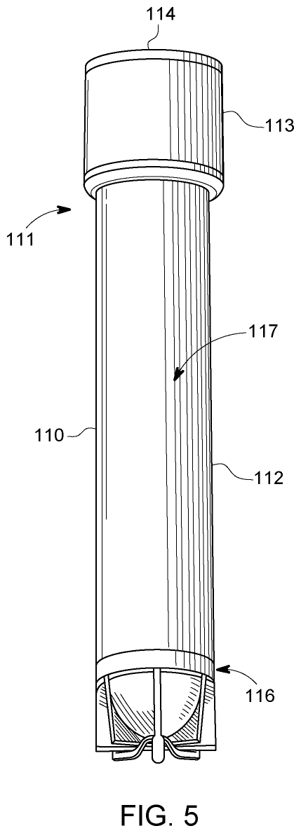

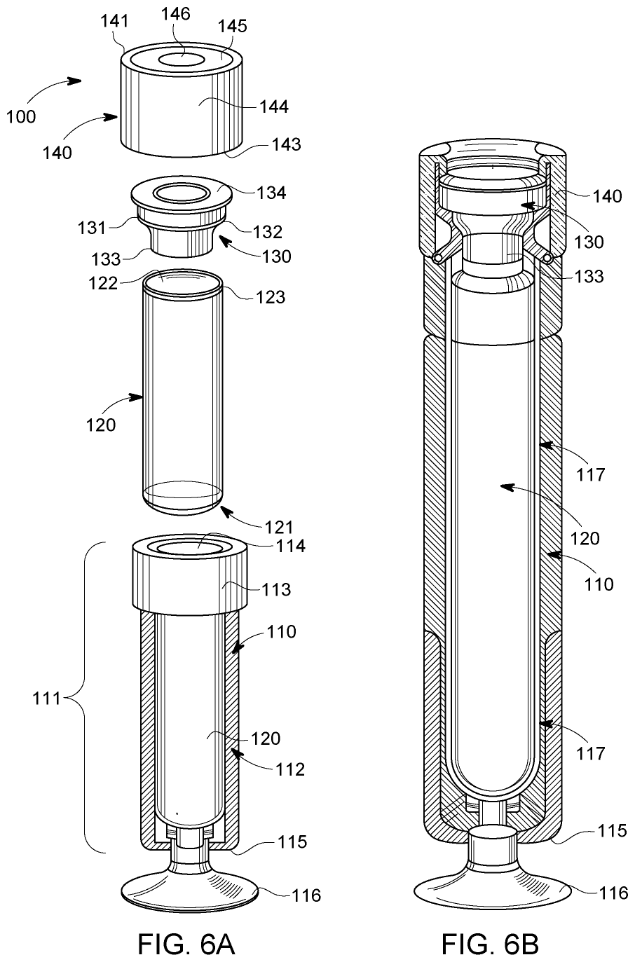

Referring now to FIG. 1 and FIG. 6A, an exploded view of the puncture-resistant protected inner container device 100 is shown comprising an outer protective body 110, an inner glass container 120, a rubber stopper 130, and an outer closure cap 140. More particularly, as shown in FIG. 5, the outer protective body 110 is in the form of an elongated tubular housing 111 having an outer surface 112, a top sleeve 113 having an open end 114, a bottom closed end 115, a stabilizing base 116 disposed at the bottom end 115, and an internal cavity 117 dimensioned to receive the inner glass container 120. The elastomeric support base 116 may be manufactured with the outer protective body 110 as a single structure. In one embodiment, the stabilizing base is in the form of a plurality of elastomeric silicone feet as shown in FIG. 5. In one embodiment the stabilizing base is in the form of a suction cup stabilization element configured to releasably engage a support surface as shown in FIG. 6B.

The inner glass container 120 comprises a closed lower end 121, an opened upper end 122, and an upper end rim 123 about the periphery of said opened upper end. The inner glass container is dimensioned to be introduced into the internal cavity 117 of the outer protective body 110. The rubber stopper 130, comprises an upper portion 131, a lower portion 133 and a stopper groove 132, the stopper groove separating the upper portion 131 from the lower portion 133. The upper portion 131 is provided with a top surface 134 and a centrally disposed puncture site 135. The rubber stopper is configured such that the lower portion 133 can be introduced into the opened upper end 122 of the inner glass container 120, the stopper groove 132 preventing the upper portion of the rubber stopper from entering the opened upper end.

The outer closure cap 140 is configured to frictionally engage the upper portion 131 of the rubber stopper 130 and to be removably coupled to the top sleeve 113 of the outer protective body 110. More specifically, the outer closure cap 140 comprises an outer cap top end 141 having an outer cap top end opening 142, an open outer cap bottom end 143, and a circular side wall 144 disposed between the cap top end 141 and the cap bottom end 143.

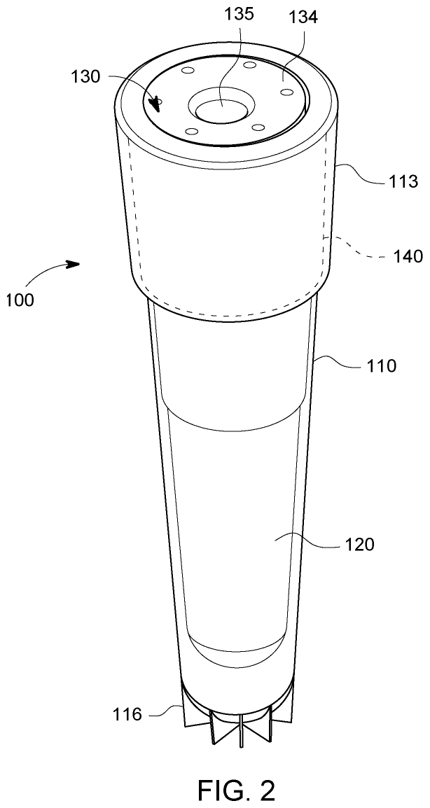

In the assembled puncture-resistant protected inner container device of the present invention, as shown in FIG. 2, the lower portion 133 of the rubber stopper 130 is introduced into the opened upper end 122 of the inner glass container, creating a fluid-tight seal and the thus-sealed inner glass container is introduced into the internal cavity 117 of the outer protective body 110. The outer closure cap 140 is configured to be frictionally introduced over the upper portion 131 of the rubber stopper 130, creating a second seal, and be removably coupled to the top sleeve 113 of the outer protective body 110 to create a third seal, such that the top surface 134 of the upper portion 131 of the rubber stopper is flush with the outer top cap end 141. This frictional contact ensures enhanced stability and contamination prevention for protecting a sample contained within the inner glass tube 120 as shown in FIG. 2. Removal of the outer closure cap 140 from the top sleeve 113 of the outer protective body causes the simultaneous extraction of the rubber stopper 130 from the inner glass container.

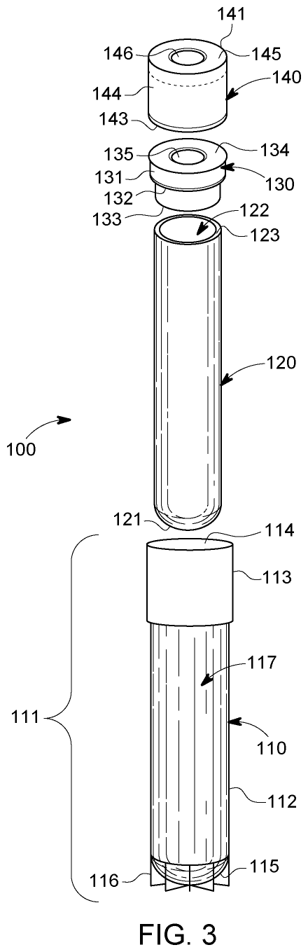

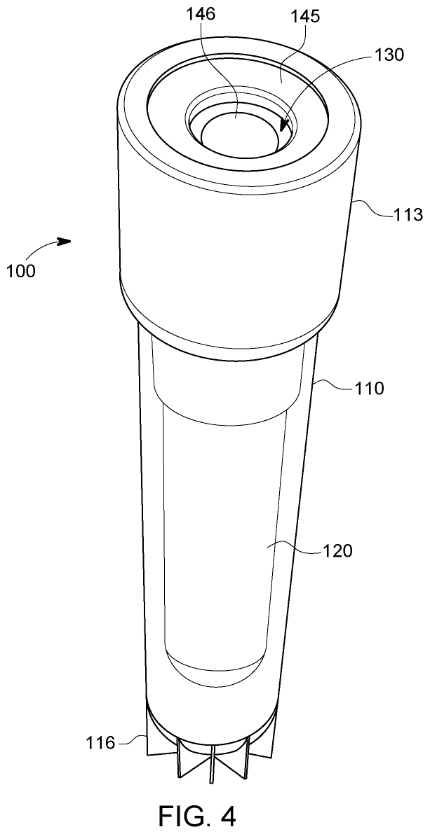

In one embodiment of the puncture-resistant protected inner container device of the present subject matter, the outer closure cap 140 is modified such that it contacts and rests upon the top surface of the rubber stopper 130. Referring to FIG. 3, the outer closure cap 140 comprises an outer cap top end 141 having an outer cap top end rim 145, an open outer cap bottom end 143, and a circular side wall 144 disposed between the cap top end 141 and the cap bottom end 143, and a centrally disposed puncture site 146.

In operation, the assembled puncture-resistant protected inner container device is shown in FIG. 2, with the lower portion 133 of the rubber stopper 130 introduced into the opened upper end 122 of the inner glass container, creating a fluid tight seal and the thus-sealed inner glass container is introduced into the internal cavity 117 of the outer protective body 110. The outer closure cap 140 is configured to be frictionally introduced over the upper portion 131 of the rubber stopper 130, creating a second seal and be removably coupled to the top sleeve 113 of the outer protective body 110 such that the outer cap top end rim 145 contacts and rests upon top surface 134 of the upper portion 131 of the rubber stopper, placing the inner container puncture site 145 in alignment with the outer cap closure puncture site 146. This frictional contact ensures enhanced stability and contamination prevention for protecting a sample contained within the inner glass container 120. Removal of the outer closure cap 140 from the top sleeve 113 of the outer protective body causes the simultaneous extraction of the rubber stopper 130 from the inner glass container.

The outer protective body is composed of a impact-resistant material which may be a molded or extruded plastic material. In one embodiment, the outer protective body is composed of an impact-absorbing polymeric or elastomeric material, such as silicone or silicone containing materials. However, it is to be understood that other materials may be suitable for use in the manufacture of the outer protective body, including, but not limited to, polyethylene terephthalate (PET), latex, ceramic, fiberglass, metal, flexi-glass, rubber, melamine, tempered glass, polypropylene, carbon, glazed stoneware, metallic glass, and combinations thereof. The material selected for making the outer protective body must be an impact-resistant material, meaning that the outer protective body can sustain an impact without damage to the inner glass container or to the biological, medical, or chemical sample contained within the inner glass tube, or to any other liquids, food, or other consumable samples.

In one embodiment, the outer protective body is translucent or transparent to allow visibility of the sample contained within the blood collection glass tube or inner glass container 120 when the inner glass container is disposed within the internal cavity 117. A transparent outer protective body provides quality control by enabling validation of the integrity of the glass tube, including determining any glass tube breakage. The puncture-resistant protected inner container device of the present subject matter provides multiple safety and quality-control advantages over the conventional breakable container systems including (1) mitigation of breakage risk during handling, transport, and centrifugation, (2) reduction of puncture or laceration injury associated with glass fracture, (3) containment of glass fragments upon accidental breakage, (4) protection of sample integrity, (5) non-destructive inspection capability, and (6) validation of glass rim integrity prior to use. Further, providing a transparent or translucent puncture-resistant outer protective body to protect an inner glass container permits selective exposure of the upper rim of the inner glass container which enables non-destructive integrity verification, thereby mitigating the risks associated with comprised vacuum seals or rim fractures as the upper rim of the inner glass container represents a critical structural zone susceptible to chips, cracks, or fracture initiation. In one embodiment, the outer protective body is composed of a silicone material adapted to be movable axially relative to the inner glass container between the protected position and a lowered position to expose the upper end of the glass tube to remove the cap and stopper to validate glass tube rim integrity for safety without making direct contact with the inner glass.

The outer protective body is designed to prevent breakage of the inner glass tube disposed in the internal cavity, thereby preventing destruction and contamination of the sample contained within the glass tube. In one embodiment, the outer protective body may be removably coupled to the inner glass container by the frictional engagement of the outer closure cap with the upper portion of the rubber stopper. In one embodiment, the outer protective body can be fixedly attached to the inner glass container for a single use. The outer protective body preferably is dimensioned to fit within a standard centrifuge and test tube holder.

The inner glass container 120, typically a blood collection tube and preferably a standard evacuated blood collection tube, is dimensioned to be introduced into the internal cavity 117 of the outer protective body 110. The inner glass container 120 is configured to receive a biological fluid, medical fluid, food sample, or chemical sample. The inner glass container preferably is composed of a non-contaminating material, preferably a non-reactive glass material, conventionally referred to as a plain glass blood collection tube. The benefit of using a non-reactive glass material is that blood coagulation is promoted within the glass collection tube, particularly when the biological, medical, or chemical sample is a blood sample. The sample collected may comprise a solid, liquid or gas. The sample is deposited into the inner container before or after the inner glass container is introduced into the inner cavity of the outer protective body, if the outer protective body is removable coupled to the inner container. Further, it is contemplated to be within the scope of the present invention that the inner glass container may further comprise at least one additive, such as an SFS coating, a gel, a clot activator, a separator, or an anticoagulant compound.

In one embodiment, the inner glass container 120 is configured to be processed in a centrifuge when assembled within the outer protective body 110. In one embodiment, the inner glass container is configured to be used in the manufacture of platelet rich fibrin (PRF). In one embodiment, the inner glass container 120 is configured to be used in In Vitro Diagnostic (IVD) tests. This frictional contact ensures enhance stability and contamination prevention for protecting the sample within the inner glass container.

The outer protective body 110 is dimensioned to receive the fluid-tight sealed inner glass container 120 as shown in FIG. 1. For illustrative purpose, the outer protective body may be about 15.0 cm in length and the inner glass container may be from about 10.0 cm to about 12.0 cm in length. In one embodiment, the outer protective body and the inner glass tube may be of equivalent lengths. In one embodiment, the inner glass container may have a length greater than that of the outer protective body, the outer closure cap being of sufficient length to engage the top end sleeve of the outer protective body. As will be obvious to those skilled in the art, conventional sizes and shapes may be implemented as necessary.

Once a sample has been collected in the inner glass container 120 and fluid-tight sealed with the rubber stopper 130, the thus-sealed inner glass container 120 is introduced into the internal cavity 117 of the outer protective body 110. The sealed inner glass container 120 is arranged in a containment position within the outer protective body by means of the frictional engagement of the upper closure cap 140 to the upper portion 131 of the rubber stopper 130 and removably coupled to the top sleeve 113 of the outer protective container 110. The configuration of the outer closure cap 140 forms an interface with the outer protective body 110. In operation, once the sealed blood collection glass tube is within the internal cavity, the outer closure cap 140 is introduced over the rubber stopper 130 and is removably coupled to the top sleeve end of outer protective body.

The outer closure cap 140 may be removably coupled to the top sleeve 113 of the outer protective body by any suitable means which creates a third seal. As shown in the Figures, the opened outer cap bottom end 143 is introduced into the top sleeve 113 and removably coupled thereto by frictional engagement. However, other types of engagement for removably coupling the outer closure cap 140 to the top sleeve 113 are contemplated to be within the scope of the present invention, including for example, threaded engagement.

Preferably, the protected inner glass container should be treated like any other collection tube or test tube with safety precautions including, for example, placing the protected device in a holder when not in use, or when placing a rubber stopper, or outer closure cap on the protected inner glass container.

The puncture-resistant protected inner container device of the present subject matter is designed to ensure that any sample container within the blood collection glass tube will not escape from inner container and into the internal cavity as a first seal is formed between the rubber stopper and the inner glass tube, a second seal is formed between the outer closure cap, the rubber stopper, and the internal glass container, and a third seal is formed between the outer closure cap and the outer protective body.

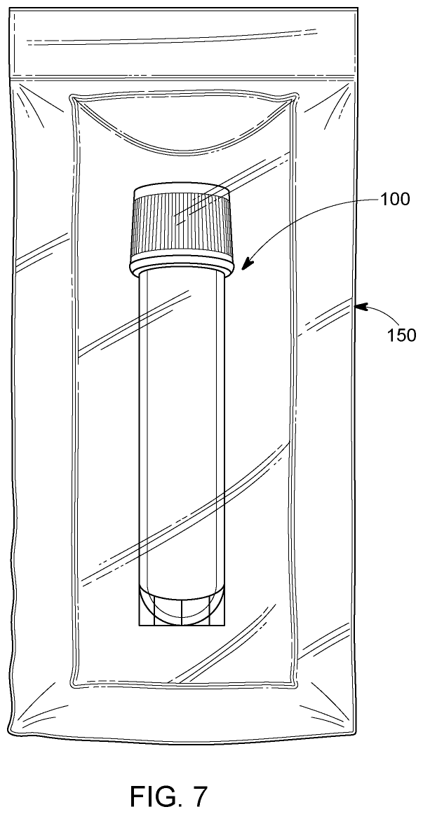

As shown in FIG. 7, the puncture-resistant protected inner container device of the present subject matter may be packaged in a single-use, sterile pack 150. In particular, the protected inner glass container can be disposed in a single sterile pack for a specific application, such as platelet-rich fibrin (PRF) applications in dental and oral surgery procedures. Specialized sterile packs may be customized according to the intended application. For example, dental and oral surgery procedures using PRF may require one to six protected devices for single use, whereas environmental applications may require a pack of twelve protected tubes for various sample sites. These sterile packs also may be used with any other medical and surgical procedures where platelet-rich fibrin applications can be beneficial.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, under the provisions of 35 U.S. C § 112, sixth paragraph, unless the element is expressly recited using the phrase “means for. . . ” and no method or process step herein is to be construed under those provisions unless the step, or steps, are expressly recited using the phrase “step(s) for. . . ”

1. A puncture-resistant protected inner container device comprising:
a. a protective outer body in the form of an elongated housing having an outer surface, a top end sleeve having a top end opening, a bottom closed end, an internal cavity, and a stabilizing base disposed on said bottom closed end;
b. an inner glass container having a closed lower end, an opened upper end, and an upper end rim about the periphery of said opened upper end, said inner container being dimensioned to fit within said internal cavity;
c. a rubber stopper having an upper portion, a lower portion, a stopper groove, said stopper groove separating said upper portion from said lower portion, said upper portion having a top surface, and a puncture site centrally disposed in said top surface, and
d. an outer closure cap having an outer cap top end, an open outer cap bottom end, and a circular side wall disposed between said outer cap top end and said open outer cap bottom end,
wherein said lower portion of said rubber stopper is frictionally introduced into said opened upper end of said internal glass container to create a first fluid-tight seal, said sealed inner glass container is introduced within said internal cavity of said protective outer body and said outer closure cap is frictionally introduced over and frictionally engages said upper portion of said rubber stopper and said opened upper end of said inner glass container to create a second seal, and is removably coupled to said top end sleeve of said protective outer body to form a third seal, and
wherein removal of said outer closure cap from said top end sleeve results in the simultaneous extraction of said rubber stopper from said inner glass container.

2. The puncture-resistant protected inner container device in accordance with claim 1, wherein said outer cap top end is an opened outer cap top end, and wherein when said outer closure cap is frictionally introduced over and frictionally engages said upper portion of said rubber stopper, said centrally disposed puncture site of said top surface of said rubber stopper is accessible for use.

3. The puncture-resistant protected inner glass container in accordance with claim 2, wherein said inner container is composed of a plain glass material.

4. The puncture-resistant protected inner container device with claim 3, wherein said inner glass container is a blood collection glass tube.

5. The puncture-resistant protected inner container device in accordance with claim 4, wherein said inner glass container is configured to be used for the separation of blood components to obtain a platelet rich fibrin clot.

6. The puncture-resistant protected inner container device in accordance with claim 1, wherein said outer protective body is composed of an impact-absorbing material.

7. The puncture-resistant protected inner container device in accordance with claim 6, wherein said impact-absorbing material is selected from the group consisting of polymeric material and elastomeric material.

8. The puncture-resistant protected inner container device in accordance with claim 7, wherein said outer protective body is composed of silicone.

9. The puncture-resistant protected inner container device in accordance with claim 6, wherein said outer protective body is composed of a material selected from the group consisting of polyethylene terephthalate and polypropylene.

10. The puncture-resistant protected inner container device in accordance with claim 6, wherein said outer protective body is a transparent outer protective body.

11. The puncture-resistant protected inner container device in accordance with claim 10, wherein said transparent outer protective body ensures quality control by enabling validation of the integrity of said inner glass container, including determining any breakage of the inner glass tube and defects of the upper end rim of the inner glass tube container.

12. The puncture-resistant protected inner container device in accordance with claim 11, wherein said transparent outer protective body is composed of a silicone material adapted to be movable axially relative to the inner glass container between a protected position and a lowered position to expose the upper end of the inner glass tube to remove the cap and stopper to validate glass tube rim integrity for safety without making direct contact with the inner glass container.

13. The puncture-resistant protected inner container device in accordance with claim 1, wherein said outer cap top end of said outer closure cap further comprises an outer cap top end rim and centrally disposed puncture site, and wherein when said outer closure cap is frictionally introduced over and frictionally engages said upper portion of said rubber stopper, said outer cap top end rim contacts and rests upon the top surface of said upper portion, with said puncture site of said rubber stopper being in alignment with said puncture side of said upper closure cap.

14. The puncture-resistant protected inner container device in accordance with claim 1, wherein said stabilizing base is selected from the group consisting of a ring-shaped support base, a suction cup stabilization element configured to releasably engage a support surface, and an elastomeric support base having a plurality of silicone feet.

15. The puncture-resistant protected inner container device in accordance with claim 14, wherein said stabilizing base is an elastomeric support base having a plurality of silicone feet.

16. The puncture-resistant protected inner container device in accordance with claim 14, wherein said stabilizing base is a suction cup stabilization element configured to releasably engage a support surface.

17. The puncture-resistant protected inner container device in accordance with claim 1, which is packaged in a single use sterile pack.

18. The puncture-resistant protected inner container device in accordance with claim 17, wherein said inner glass container is configured to be used for the separation of blood components to obtain a platelet rich fibrin clot.

19. The puncture-resistant protected inner container device in accordance with claim 17, wherein said inner glass container is a blood collection glass tube.

20. The puncture-resistant protected inner container device in accordance with claim 18, wherein said inner glass container is configured to be used for the separation of blood components to obtain a platelet rich fibrin clot (PRF) for application in dental and oral surgery procedures, and any medical and surgical procedures where the application of PRF can be beneficial.
